(19) United States
(12) Patent Application Publication
(10) Pub. No.: US 2013/0052275 A1
(43) Pub. Date: Feb. 28, 2013

(54) Warming Inch Loss Delivery System

(71) Applicant: Michelle Ward, Elkins Park, PA (US)

(72) Inventor: Michelle Ward, Elkins Park, PA (US)

(73) Assignee: Michelle Ward, Elkins Park, PA (US)

(21) Appl. No.: 13/592,285

(22) Filed: Aug. 22, 2012

Related U.S. Application Data
(60) Provisional application No. 61/526,897, filed on Aug. 24, 2011.

(51) Int. Cl. A61K 36/9068; A61K 33/26; A61K 33/30; A61K 33/24; A61K 33/32; A61P 17/00; A61K 36/8962; A61K 36/9066; A61K 36/81; A61K 36/31; A61P 3/04; A61K 36/54; A61K 33/42

(52) U.S. Cl. 424/601; 424/739; 424/682; 424/646; 424/600; 424/642; 424/639; 424/655; 424/754; 424/756; 424/760; 424/755

(57) ABSTRACT

This invention is a topical treatment for the skin, used for skin enhancement, firming, smoothing, refining and to reduce the appearance of cellulite; that has a self-warming or thermogenic effect comprised of a base combination of warming herbs (Ayurvedic), being any herbs that create a warming effect, combined with dietary minerals that can be blended together in any ratio.

This application claims benefit of U.S. Provisional Application No. 61/526,897, filed on Aug. 24, 2011.

### STATEMENT REGARDING FEDERALLY SPONSORED RESEARCH AND DEVELOPMENT

N/A

### BACKGROUND OF THE INVENTION

This invention relates to a cosmetic composition that creates a slimming and skin enhancing effect which may include an inch loss measured by the circumference of a body part(s); a skin refining which may include the look of firmness and an improvement in the appearance of cellulite on the topical layer of the skin in any area that the composition has been correctly applied.

Existing topical creams on the market lack an effectiveness and ease of use that this invention brings to the marketplace. This is due largely to fact that these other compositions do not have a self-warming component. Current ‘inch loss’ or skin firming products often lack a warming component which causes all or most of the product to lay on the surface of the skin and not penetrate that top layer of the skin greatly, if at all. They often require an outside heating element to warm them or an apparatus to drive the composition into the epidermis. Also, other topical skin refining compositions lack a natural yet highly effective active ingredient such as the dietary minerals that this invention includes in order to bring a benefit to the body, through the skin, and a noticeable skin enhancement. It is this combination of the dietary minerals and the warming, or otherwise considered ‘Ayurvedic Herbs’, which makes this invention unique and self-contained in that no outside warming or mechanics are necessary for its effectiveness of use.

### SUMMARY OF THE INVENTION

The present invention is that of a slimming or inch loss topical cosmetic composition for the body consisting of a combination of dietary minerals and warming, or otherwise called ‘Ayurvedic Herbs’, in any ratio. This blend of warming herbs and dietary minerals allows the composition to self-warm (due to the herbs) allowing the minerals to enter the epidermis and create a skin enhancing, firming effect.

### DETAILED DESCRIPTION OF THE INVENTION

The present invention is a delivery system wherein other components can be added to the base combination of warming herbs and dietary minerals to create different versions of the same cosmetic, skin enhancing composition. The base combination, or delivery system, is comprised of warming (Ayurvedic) herbs and dietary minerals combined in any ratio. This combination in a cosmetic composition allows for a noticeable skin enhancement that could include a reduction in the appearance of cellulite, a tightening or firming of the skin and an inch loss of the circumference of a properly treated area.

The presence of the dietary minerals are included to allow the body to balance out disproportioned mineral levels on a temporary basis so that the waste fluids that are logged in and around adipose tissues can be released during normal elimination. This release can create an inch loss and skin refining in the properly treated area(s) because the said fluids are no longer present. No dehydration occurs and this fluid is actually gone. As the body created the said fluid, new fluids will accumulate and be stored. Maintenance usage of this invention can help to control buildup of said fluid which helps with the appearance of the skin and is overall a healthy option for the body.

The presence of the warming herbs, or otherwise considered ‘Ayurvedic Herbs’, are included to prepare the top layer of the skin (epidermis) thereby relaxing and opening the pores of said skin. This enables the cosmetic composition to penetrate past the outer barrier of the skin and address fluids that lay beneath that layer and create a slackened appearance.

The two ingredient groups, being the warming herbs and the dietary minerals work together to act as a ‘driver’ and an active ingredient to release the said fluids.

The invention claimed is:

1. A topical, cosmetic composition that is comprised of a combination of warming, or Ayurvedic, herbs and dietary minerals that will be combined in any ratio; which results in a skin enhancing, inch loss and/or reduction in the appearance of cellulite.

2. The cosmetic composition of claim 1, wherein said composition is in a form selected from the group consisting of a cream, a milk, a milky lotion, a gel, a patch, a paste and/or a spray.

3. A cosmetic treatment for improving the esthetic appearance of a person comprising applying to the skin of said person the cosmetic slimming composition of claim 1.

4. The composition of claim 1, wherein said warming herbs are taken from a group consisting of cayenne, cinnamon, turmeric, cumin, capsicum, cardamom, ginger, fennel, clove, mustard, paprika, garlic and others.

5. The composition of claim 1, wherein said dietary minerals are taken from a group consisting of magnesium, calcium, chloride, iron, copper, potassium, selenium, zinc, sodium, molybdenum, manganese, chromium, phosphorous and others.
